(19) United States
(12) Patent Application Publication
(10) Pub. No.: US 2014/0216444 A1
(43) Pub. Date: Aug. 7, 2014

(54) METERED DOSE INHALER WITH AN ELECTRONIC DOSE COUNTER

(71) Applicant: Flextronics AP, LLC, San Jose, CA (US)

(72) Inventors: Lior Shtram, Tel Aviv (IL); Yossef Schvetz, Milano (IT); Serdar Ozsumer, Milano (IT); Marco Trecate, Milano (IT); Livia Ardissone, Monza (MB) (IT); Aurelio Bonfanti, Lissone (MI) (IT); Marco Vergani, Seregeno (MB) (IT); Paola Nonini, Monza (MB) (IT); Nisse van der Velde, Monza (MB) (IT)

(73) Assignee: FLEXTRONICS AP, LLC, San Jose, CA (US)

(21) Appl. No.: 14/171,918

(22) Filed: Feb. 4, 2014

Related U.S. Application Data
(60) Provisional application No. 61/761,500, filed on Feb. 6, 2013.

(51) Int. Cl. A61M 15/00

(52) U.S. Cl. CPC A61M 15/008; A61M 15/009; A61M 15/0025
USPC 128/200.23; 29/428

(57) ABSTRACT

A metered dose inhaler with an electronic dose counter, and method of operating and forming the same. In one embodiment, the metered dose inhaler includes a pressurized canister assembly formed with a memory device affixed thereto. The metered dose inhaler also includes a canister housing into which the pressurized canister assembly is inserted, the canister housing formed with an electronic dose counter including a display configured to display a dose count of a drug in the pressurized canister assembly stored in the memory device.

This application claims the benefit of U.S. Provisional Application No. 61/761,500, entitled “Inhaler,” filed on Feb. 6, 2013, which application is incorporated herein by reference.

### TECHNICAL FIELD

The present invention is directed, in general, to medical drug inhalers and, more specifically, to a metered dose inhaler and method of operating the same.

### BACKGROUND

In medical applications, a drug is administered to a patient by oral inhalation of the drug to treat symptoms of upper respiratory diseases such as asthma and chronic obstructive pulmonary disease. A drug delivery device is formed with a pressurized canister containing a non-toxic pressurized gas and typically a finely powdered form of the drug. The pressurized canister is inserted into a canister housing formed with a mouthpiece and an aperture configured to receive a discharge port of the canister. The mouthpiece is inserted by the patient into the mouth, and the patient inhales while depressing the pressurized canister into the canister housing, which causes a metered dose of the drug retained in the pressurized canister to be dispensed into the patient's mouth and into adjacent upper respiratory passages. The pressurized canister is formed with a suitable structure to discharge the metered dose when the pressurized canister is depressed into the canister housing.

The pressurized canister is initially charged with a measured quantity of the pressurized gas and the powdered drug, which provides a predetermined number of metered drug dosages (e.g., 200 metered doses). The metered doses are administered to the patient by repeated depressions of the pressurized canister into the canister housing. However, there is generally no indication to the patient or to a caregiver of the number of doses that the patient has inhaled or the number of doses remaining in the pressurized canister. Careful record-keeping by the patient or the caregiver is generally not a reliable process for a typical patient, who may have reduced physical and mental skills due to aging or disease to track the number of doses already administered or are remaining in the pressurized canister.

Accordingly, what is needed in the art is a process and method to provide an indication to a patient and/or a caregiver of the number of discharged and/or remaining doses in the pressurized canister.

### SUMMARY OF THE INVENTION

Technical advantages are generally achieved, by advantageous embodiments of the present invention, including a metered dose inhaler with an electronic dose counter, and method of operating and forming the same. In one embodiment, the metered dose inhaler (“MDI”) includes a pressurized canister assembly formed with a memory device affixed thereto. The MDI also includes a canister housing into which the pressurized canister assembly is inserted, the canister housing formed with an electronic dose counter including a display configured to display a dose count of a drug in the pressurized canister assembly stored in the memory device.

The foregoing has outlined rather broadly the features and technical advantages of the present invention in order that the detailed description of the invention that follows may be better understood. Additional features and advantages of the invention will be described hereinafter, which form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present invention, reference is now made to the following descriptions taken in conjunction with the accompanying drawings, in which:

FIG. 1 illustrates a view of an embodiment of an MDI;

FIG. 2 illustrates a view of an embodiment of a pressurized canister assembly of FIG. 1;

FIG. 3 illustrates a view of an embodiment of a pressurized canister assembly and a canister housing of FIGS. 1 and 2;

FIG. 4 illustrates a block diagram of an embodiment of an electronic dose counter embodied in an MDI; and

FIG. 5 illustrates a flow diagram of an embodiment of a method of forming an MDI.

Corresponding numerals and symbols in the different figures generally refer to corresponding parts unless otherwise indicated, and may not be redescribed in the interest of brevity after the first instance. The FIGURES are drawn to illustrate the relevant aspects of exemplary embodiments.

### DETAILED DESCRIPTION OF ILLUSTRATIVE EMBODIMENTS

The making and using of the present exemplary embodiments are discussed in detail below. It should be appreciated, however, that the present invention provides many applicable inventive concepts that can be embodied in a wide variety of specific contexts. The specific embodiments discussed are merely illustrative of specific ways to make and use the invention, and do not limit the scope of the invention.

The present invention will be described with respect to exemplary embodiments in a specific context, namely, a metered dose inhaler (“MDI”). While the principles of the present invention will be described in the environment of a medical application, any application that may benefit from a device that provides an inhalable or otherwise discharged metered dose is well within the broad scope of the present invention.

A problem with a conventional metered dose inhaler charged with an original drug quantity is that it provides no visual indication to a patient like countable pills, which a patient or caregiver can visually examine to determine how many doses are left. As introduced herein, an MDI is enhanced with an electronic dose counter to provide an improved drug inhaling device that can be employed to reduce misuse of drug administration from a patient's or caregiver's perspective.

The MDI is formed with a memory device (e.g., a semiconductor memory device) affixed to a pressurized canister assembly (e.g., a disposable pressurized canister assembly). The pressurized canister assembly is formed with a pressurized canister and a canister cap attached thereto. The memory device is coupled to a processor formed in a canister housing to enable the canister housing to operate as a durable dose counter.

The MDI can be constructed with several pieces. A mouthpiece (e.g., a disposable mouthpiece) is coupled to a canister housing configured to hold a sensor for sensing administration of a drug dose. The MDI also includes a processor, a battery and a display. In an embodiment, the display exhibits remaining dose count and other information such as a date, a time, and a warning, not necessarily at the same moment of time. The MDI also includes a pressurized canister assembly that includes a memory device (e.g., a semiconductor memory device) affixed to a canister cap. The processor is constructed with an electronic dose counter that counts backwards to zero from an original dose count (e.g., 200 inhalable doses) that is charged into the MDI.

In an embodiment, the MDI is constructed so that the processor and the battery are located in the canister housing to be reused with many pressurized canister assemblies. However, the memory device itself which maintains the remaining dose count is associated with, and is permanently attached to, a particular pressurized canister. A small memory device can be attached to a pressurized canister via the canister cap. The MDI includes a sensor that, in an embodiment, can be located in the canister housing that detects a discharge of individual doses and decrements via the processor the remaining dose count in the memory device as doses are discharged.

Because a battery can eventually become fully discharged, which can compromise the content of a random-access semiconductor memory device, a detector (e.g., a weak/discharged battery detector) is provided that senses an open-circuit voltage of the battery or a count of drug discharges and provides a warning to a patient if complete or substantial discharge of the battery is imminent. In such a case, the patient is warned in sufficient time to obtain another canister housing in which a new battery is furnished. Substantial discharge may be an estimate that a fraction such as 80 percent of the battery has been discharged.

The memory device may be a flash memory device that does not require the battery to maintain its memory content. The display, which can be a liquid crystal display, can be affixed to the canister housing. In an embodiment, the mouthpiece and a mouthpiece cap, which are inexpensive items to produce, are disposable, but the canister housing is constructed so that it can be reused with many pressurized canister assemblies. The structure enables a low-cost disposable pressurized canister assembly instead of increasing the cost of the pressurized canister assembly by attaching the electronic dose counter including the sensor, processor and battery thereto. Communication and powering between the electronics in the canister housing and the memory device can be performed by wireless data transmission such as a radio frequency identification (“RFID”) device or with metallic contacts.

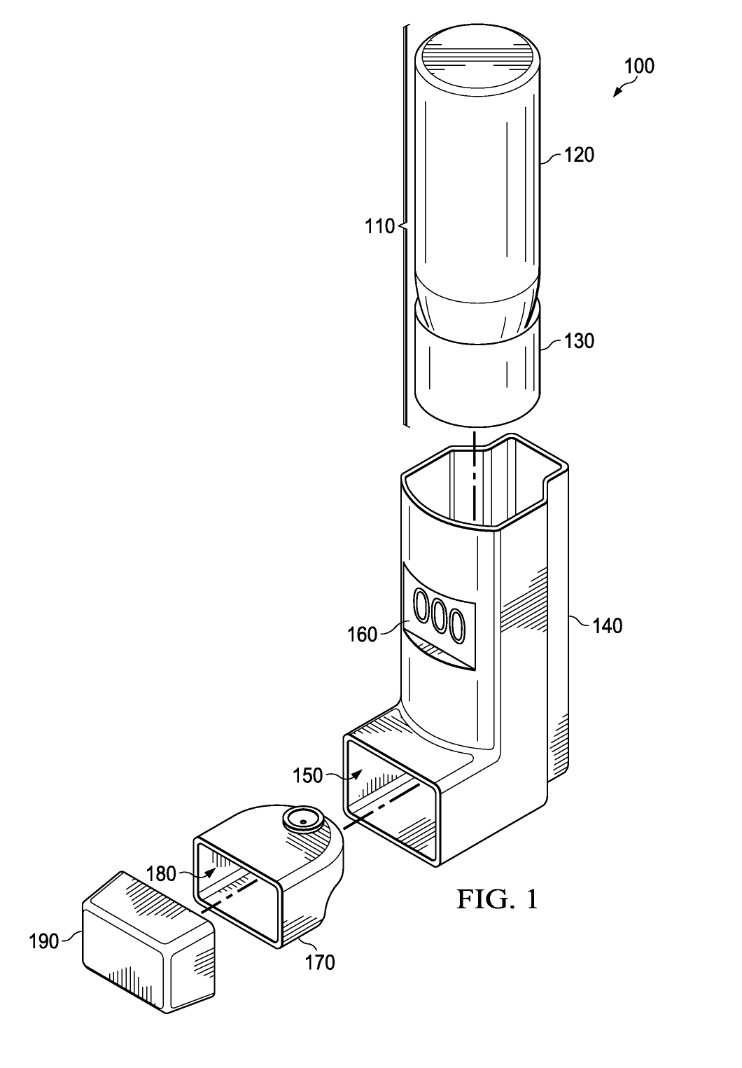

Referring initially to FIG. 1, illustrated is a view of an embodiment of an MDI 100. The MDI 100 is constructed with a pressurized canister assembly (e.g., a disposable pressurized canister assembly) 110 formed with a pressurized canister 120 that is pressurized with a non-toxic gas and a finely powdered form of the drug that can be safely inhaled by a patient. The pressurized canister 120 is fitted with a canister cap 130. The pressurized canister assembly 110 is constructed so that it can be inserted into a canister housing (e.g., a durable canister housing) 140 which can be formed with a drug-delivery aperture 150 and a display 160. A mouthpiece (e.g., a disposable mouthpiece) 170 is formed so that it can be inserted into or over the drug-delivery aperture 150 and includes a drug inhaling aperture 180. An inhaler cap 190 can be fitted into or over the mouthpiece 170.

In operation, the pressurized canister assembly 110, after insertion into the canister housing 140, is depressed to produce an inhalable, metered dose through the drug inhaling aperture 180 of the mouthpiece 170 for inhalation by a patient by inserting the mouthpiece 170 into the mouth. The patient inhales the metered dose through the drug inhaling aperture 180 while depressing the pressurized canister assembly 110. The number of remaining doses and other information of interest to the patient and/or a caregiver such as dates and times of previously administered doses and state of the battery is displayed on a display 160.

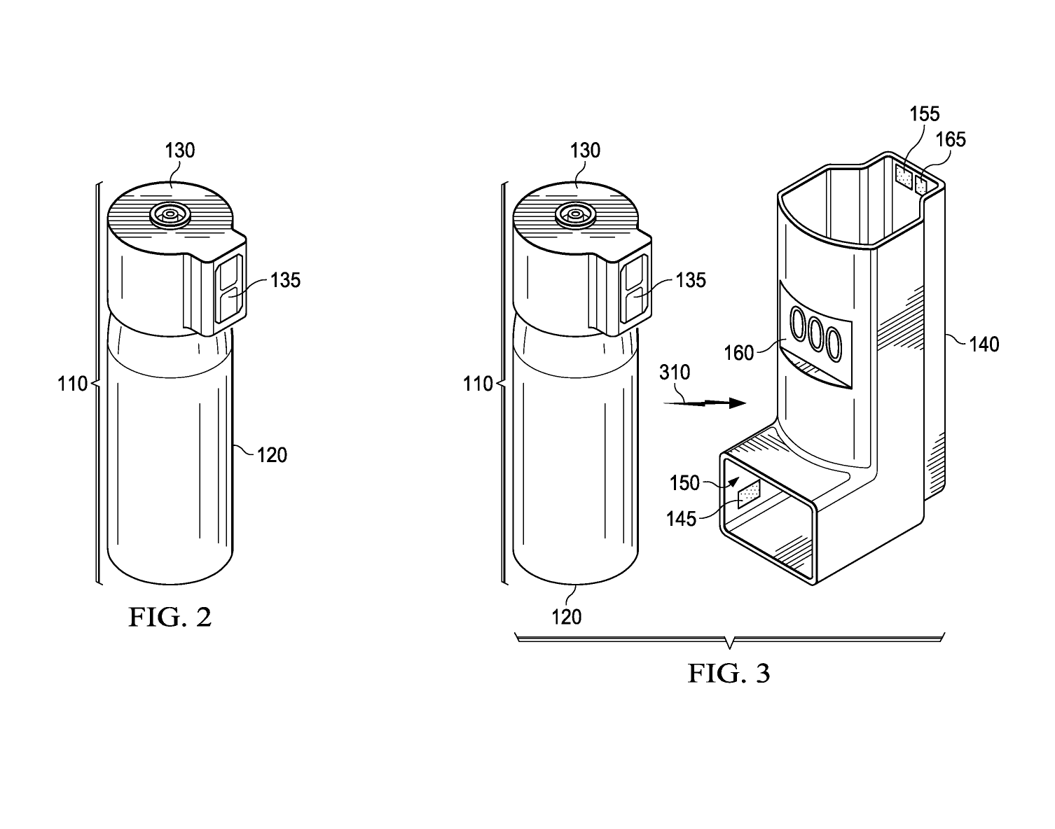

Turning now to FIG. 2, illustrated is a view of an embodiment of the pressurized canister assembly 110 of FIG. 1. The pressurized canister assembly 110 includes the canister cap 130 fitted onto the pressurized canister 120. The canister cap 130 is constructed with a memory device 135 that can be a flash memory device. While the canister cap 130 is typically retained with the pressurized canister 120, the canister cap 130 can be disassembled with some effort from the pressurized canister 120 so that the pressurized canister 120 can be disposed of while retaining dose information stored in the memory device 135. The canister cap 130 can also be disposed of or retained for later assessment of previously administered doses by a physician or a caregiver.

Turning now to FIG. 3, illustrated is a view of an embodiment of the pressurized canister assembly 110 and the canister housing 140 of FIGS. 1 and 2. The pressurized canister assembly 110 is able to communicate over a wireless data transmission path 310 with the canister housing 140 so that the remaining dose count and/or other information can be displayed to the patient and/or a caregiver on the display 160. In an alternative embodiment, the pressurized canister assembly 110 is configured to communicate over a conductive metallic path with the canister housing 140. The canister housing 140 is formed with a sensor 145 configured to detect a discharge of a metered dose. The sensor 145 is coupled to a processor 155 in the canister housing 140 to enable the processor 155 to numerically track/count the number of doses discharged from the pressured canister 120 of the pressurized canister assembly 110, thereby to maintain a count of the doses remaining in the pressured canister 120 of the pressurized canister assembly 110. A power source (e.g., a battery) with a detector 165 powers an electronic dose counter (including the memory device 135, the sensor 145, the processor 155 and the display 160) and monitors a state of the battery, respectively.

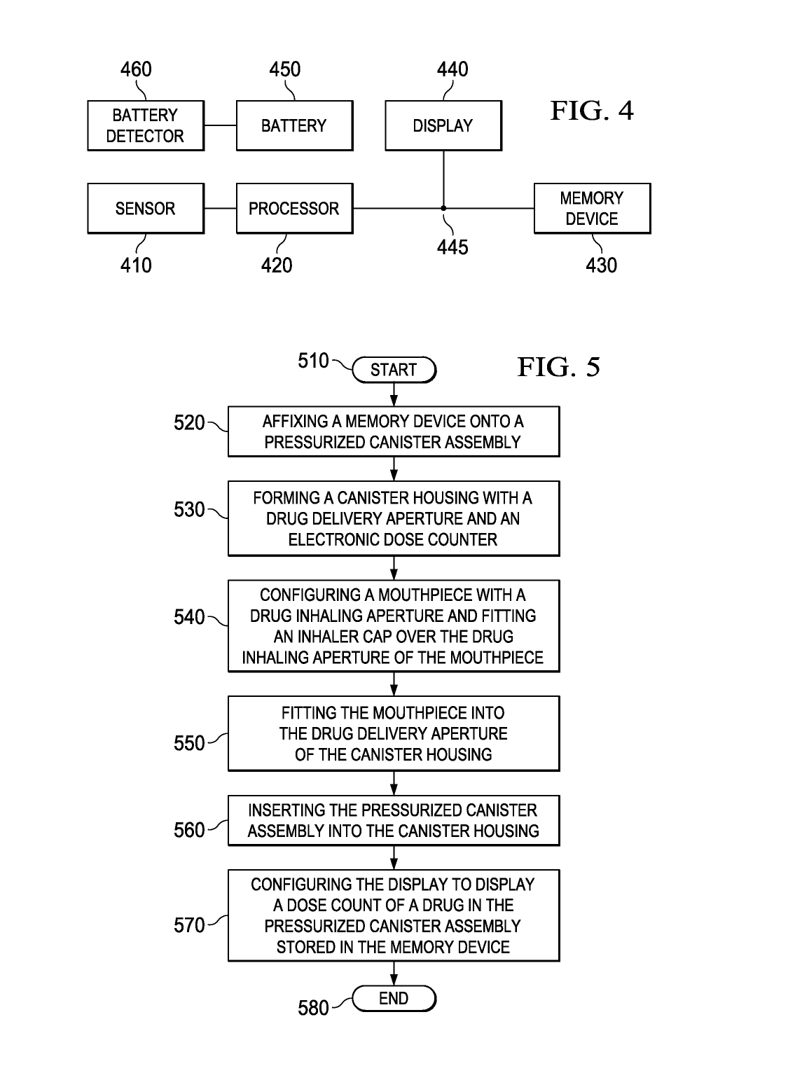

Turning now to FIG. 4, illustrated is a block diagram of an embodiment of an electronic dose counter embodied in an MDI. The electronic dose counter includes a sensor 410, a processor 420, a memory device 430 and a display 440. The sensor 410 detects a discharge of individual doses and provides a signal to the processor 420. Responsive to the signal, the processor 420 numerically tracks/counts the number of doses discharged from a pressurized canister assembly by, for instance, decrementing the remaining dose count in the memory device 430 as doses are discharged from the pressurized canister assembly. The processor 420 and memory device 430 cooperate to provide a signal (e.g., over a metallic path 445 with metallic contacts) to the display 440 to display a remaining dose count of the pressurized canister assembly that is stored in the memory device 430. A power source (e.g., a battery) 450 powers the electronic dose counter and a detector (e.g., a battery detector) 460 monitors a state (e.g., discharge status) of the battery 450.

Turning now to FIG. 5, illustrated is a flow diagram of an embodiment of a method of forming an MDI. The method begins in a start step or module 510. In a step or module 520, a memory device is affixed onto a pressurized canister assembly including a pressurized canister and a canister cap. In a step or module 530, a canister housing is formed with a drug delivery aperture and an electronic dose counter including a sensor, a processor, a battery and a display. In a step or module 540, a mouthpiece with a drug inhaling aperture is configured, and an inhaler cap is fitted over the drug inhaling aperture of the mouthpiece. In a step or module 550, the mouthpiece is fitted into the drug delivery aperture of the canister housing. In a step or module 560, the pressurized canister assembly is inserted into the canister housing. In a step or module 570, the display is configured to display a dose count of a drug in the pressurized canister of the pressurized canister assembly that is stored in the memory device. The method ends in step or module 580. Of course, the sequence of design of forming the MDI may be altered depending on the application.

The MDI with the electronic dose counter or related method of operating the same may be implemented as hardware (embodied in one or more chips including an integrated circuit such as an application specific integrated circuit), or may be implemented as software or firmware for execution by a processor (e.g., a digital signal processor) in accordance with memory. In particular, in the case of firmware or software, the exemplary embodiment can be provided as a computer program product including a computer readable medium embodying computer program code (i.e., software or firmware) thereon for execution by the processor.

Program or code segments making up the various embodiments may be stored in the computer readable medium. For instance, a computer program product including a program code stored in a computer readable medium (e.g., a non-transitory computer readable medium) may form various embodiments. The “computer readable medium” may include any medium that can store or transfer information. Examples of the computer readable medium include an electronic circuit, a semiconductor memory device, a read only memory (“ROM”), a flash memory, an erasable ROM (“EROM”), a floppy diskette, a compact disk (“CD”)-ROM, and the like.

Those skilled in the art should understand that the previously described embodiments of an MDI with an electronic dose counter and related methods of forming/operating the same are submitted for illustrative purposes only. An MDI as described hereinabove may also be applied to other systems such as, without limitation, a device for applying or spraying a gas-transported substance to an external surface, such as an external surface of the body or of a mechanical part.

Also, although the present invention and its advantages have been described in detail, it should be understood that various changes, substitutions and alterations can be made herein without departing from the spirit and scope of the invention as defined by the appended claims. For example, many of the processes discussed above can be implemented in different methodologies and replaced by other processes, or a combination thereof.

Moreover, the scope of the present application is not intended to be limited to the particular embodiments of the process, machine, manufacture, composition of matter, means, methods, and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A metered dose inhaler (MDI), comprising:
a pressurized canister assembly formed with a memory device affixed thereto; and
a canister housing into which said pressurized canister assembly is inserted, said canister housing formed with an electronic dose counter including a display configured to display a dose count of a drug in said pressurized canister assembly stored in said memory device.

2. The MDI as recited in claim 1 further comprising a mouthpiece formed with a drug inhaling aperture and configured to be fitted into a drug delivery aperture of said canister housing.

3. The MDI as recited in claim 2 further comprising an inhaler cap configured to be fitted over said drug inhaling aperture of said mouthpiece.

4. The MDI as recited in claim 1 wherein said pressurized canister assembly includes a pressurized canister and a canister cap.

5. The MDI as recited in claim 4 wherein said memory device is affixed to said canister cap.

6. The MDI as recited in claim 4 wherein said canister cap is fitted to said pressurized canister.

7. The MDI as recited in claim 1 wherein said memory device communicates with said electronic dose counter over a wireless data transmission path or a metallic path with metallic contacts.

8. The MDI as recited in claim 1 wherein said pressurized canister assembly is configured to discharge a metered dose of said drug contained in a pressured canister as a powder and a non-toxic pressurized gas.

9. The MDI as recited in claim 1 wherein said electronic dose counter comprises a sensor and processor configured to cooperate with said memory device to provide said dose count to said display.

10. The MDI as recited in claim 1 wherein said electronic dose counter comprises a battery configured to power said electronic dose counter and a detector configured to provide a status of said battery.

11. A method of forming a metered dose inhaler (MDI), comprising:
providing a pressurized canister assembly;
affixing a memory device to said pressurized canister assembly; and
inserting said pressurized canister assembly into a canister housing, said canister housing formed with an electronic dose counter including a display configured to display a dose count of a drug in said pressurized canister assembly stored in said memory device.

12. The method as recited in claim 11 further comprising forming a mouthpiece with a drug inhaling aperture and fitting said mouthpiece into a drug delivery aperture of said canister housing.

13. The method as recited in claim 12 further comprising fitting an inhaler cap over said drug inhaling aperture of said mouthpiece.

14. The method as recited in claim 11 wherein said pressurized canister assembly includes a pressurized canister and a canister cap.

15. The method as recited in claim 14 wherein said memory device is affixed to said canister cap.

16. The method as recited in claim 14 further comprising fitting said canister cap to said pressurized canister.

17. The method as recited in claim 11 wherein said memory device communicates with said electronic dose counter over a wireless data transmission path or a metallic path with metallic contacts.

18. The method as recited in claim 11 wherein said pressurized canister assembly is configured to discharge a metered dose of said drug contained in a pressured canister as a powder and a non-toxic pressurized gas.

19. The method as recited in claim 11 wherein said electronic dose counter comprises a sensor and processor configured to cooperate with said memory device to provide said dose count to said display.

20. The method as recited in claim 11 wherein said electronic dose counter comprises a battery configured to power said electronic dose counter and a detector configured to provide a status of said battery.
